(19) United States
(12) Plant Patent Application Publication
(10) Pub. No.: US 2012/0102603 P1
(43) Pub. Date: Apr. 26, 2012

(54) Physalis plant named 'Queen'

(50) ;

(75) Inventor: Georg Uebelhart, Schwarmstedt (DE)

(73) Assignee: Amazing Breeders Company B.V., Aalsmeer (NL)

(21) Appl. No.: 12/925,425

(22) Filed: Oct. 21, 2010

(51) Int. Cl. A01H 5/00

(52) U.S. Cl. PLT/263.1

(57) ABSTRACT

This invention relates to a new and distinct variety of Physalis plant named ‘Queen’. The new variety is primarily characterized by an upright, very vigorous growth habit and bright orange husks, is disclosed.

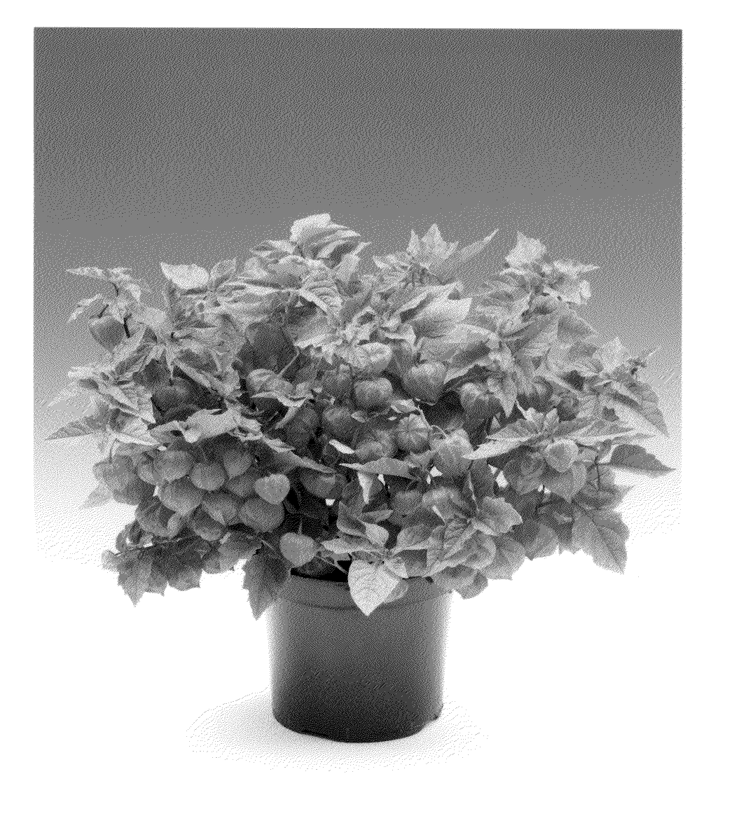

### GENUS AND SPECIES

Physalis alkekengi

### VARIETY DENOMINATION

‘Queen’

### BACKGROUND OF THE NEW PLANT

The present invention relates to a new and distinct Physalis variety designated ‘Queen’ and botanically known as Physalis alkekengi. This new Physalis variety was discovered in Toggraben, Germany in September 2007 and originated from an open pollination between the proprietary female parent ‘06-31’ (unpatented) and un-named Physalis plants. The original seedling of the new variety was first asexually propagated in Boijl and Aalsmeer, The Netherlands in 2008.

‘Queen’ was subsequently asexually propagated and underwent further testing in Boijl and Aalsmeer, The Netherlands for two years. The present invention has been found to retain its distinctive characteristics through successive asexual propagations via stolons and tissue culture.

Plant Breeder's Rights for this variety have not been applied for. ‘Queen’ has not been made publicly available or sold more than one year prior to the filing date of this application.

### DESCRIPTION OF THE PHOTOGRAPH

The accompanying color photograph shows the overall plant habit of a 6-month old plant. The colors shown are as true as can be reasonably obtained by conventional photographic procedures.

### DESCRIPTION OF THE NEW VARIETY

The following detailed descriptions set forth the distinctive characteristics of ‘Queen’. The data which define these characteristics is based on observations taken in Boijl and Aalsmeer, The Netherlands in September 2010. This description is in accordance with UPOV terminology. Color designations, color descriptions, and other phenotypical descriptions may deviate from the stated values and descriptions depending upon variation in environmental, seasonal, climatic, and cultural conditions. ‘Queen’ has not been observed under all possible environmental conditions. The botanical description of ‘Queen’ was taken from 6 month old plants. Color terminology follows The Royal Horticultural Society Colour Chart, London (RHS) (2001).

### DETAILED BOTANICAL DESCRIPTION OF THE PLANT

Classification:
   Species.—Physalis alkekengi.
   Common name.—Physalis.
   Denomination.—‘Queen’.
Plant:
   Height (cm).—40.0 cm.
   Diameter (cm).—30.0.
   Habit and shape.—Upright.
   Vigor.—Very vigorous.
Shoot:
   Attitude of one year old shoot.—40.0 cm.
   Curvature of one year old shoot.—Straight.
   Number of basal shoots per plant.—30.
   Prickles.—Absent.
   Color.—RHS 147C.
Leaves:
   Length, mature (cm).—8.0 cm.
   Width, mature (cm).—5.0 cm.
   Apex.—Acute.
   Base.—Obtuse.
   Margin.—Entire.
   Shape.—Deltoid/aristate.
   Color, both immature and mature.—Upper surface: RHS 146A Lower surface: RHS 146B.
   Texture.—Smooth.
   Anthocyanin.—Absent.
   Angle of base of blade with petiole.—Obtuse.
   Glossiness of both leaf surfaces.—Weak.
   Petiole.—Length (cm): 4.0 cm Diameter (cm): 4.0 mm Pubescence: Absent Color: RHS 146A.
Flower:
   Predominant number of flowers per plant.—Greater than 50.
   Time of flowering (from first to last flower).—July.
   Sepal.—Shape: Ovate/rounded and fused Base: Fused Length: 5.0 cm Width: 3.0 cm Number of sepals per flower: 5 Color (both upper and lower surfaces): RHS 30A.
Fruit:
   Length.—1.0 cm.
   Width.—1.0 cm.
   Weight, per individual fruit.—10 g.
   Relative fruit size.—Small.
   Predominant fruit shape.—Round.
   Color.—RHS 44B.
   Evenness of fruit color.—Even.
   Pubescence.—Absent.
   Veining.—Strong.
   Fruit glossiness.—High.
   Firmness of flesh.—Firm.
   Color of flesh.—RHS 44B.
   Evenness of flesh color.—Even.
   Peduncle.—Length (cm): 3 cm Diameter (cm): 1 mm Color: RHS 146A Pubescence: absent.
Disease and insect resistance: No particular resistance observed.

### COMPARISON WITH PARENTAL VARIETY

Physalis ‘Queen’ cannot be compared to the female parent ‘06-31’, because plants of ‘06-31’ are no longer available.

I claim:

1. A new and distinct variety of Physalis plant as described and shown herein.
